(19) United States
(12) Patent Application Publication
(10) Pub. No.: US 2004/0064099 A1
(43) Pub. Date: Apr. 1, 2004

(54) Intraluminal needle injection substance delivery system with filtering capability

(76) Inventor: Jessica G. Chiu, Belmont, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/260,742

(22) Filed: Sep. 30, 2002

(51) Int. Cl. A61M 5/178

(52) U.S. Cl. 604/164.01; 606/200

(57) ABSTRACT

An intraluminal needle injection substance delivery system includes a delivery catheter having a delivery lumen extending therethrough to provide a conduit by which a drug delivery device can extend through to inject a therapeutic drug or substance to an area of treatment in a body vessel. The delivery system includes filtering capability for capturing possible unwanted debris which may be entrained in the patient's vasculature. The filtering device used in accordance with the delivery catheter may include a filtering element placed downstream from the area in which the therapeutic drug or substance is to be administered. The delivery lumen can be attached to the filtering device for deployment within the patient's vasculature. In this fashion, as the filtering device is expanded from a collapsed, delivery position to an expanded position within the patient's vasculature, the end of the delivery lumen will be positioned near or in contact with the target area in the patient's vasculature. A substance delivery device, such as a syringe having an elongated cannula, can be placed in the delivery lumen to inject the vessel wall with a drug or other therapeutic substance.

### BACKGROUND OF THE INVENTION

The present invention relates generally to medical devices and systems for administering therapeutic substances, such as therapeutic agents and drugs, in a vascular lumen of a patient's anatomy. More particularly, the present invention is directed to a local delivery system which utilizes a catheter for administering a therapeutic substance intraluminally and includes filtering capability for capturing large particles or microspheres of the therapeutic substance or ruptured vulnerable plague which could otherwise enter the patient's vasculature and possibly block vital body vessels located downstream from the area of treatment. Additionally, the present invention is directed to methods for delivering therapeutic substances into a patient's vasculature while providing filtering capability.

Numerous procedures have been developed for treating occluded blood vessels to allow blood to flow without obstruction. Such procedures may involve the percutaneous introduction of an interventional device into the lumen of the artery, usually by a catheter. One widely known and medically accepted procedure is balloon angioplasty in which an inflatable balloon is introduced within the stenosed region of the blood vessel to dilate the occluded vessel. The balloon dilatation catheter is initially inserted into the patient's arterial system and is advanced and manipulated into the area of stenosis in the artery. The balloon is inflated to compress the plaque and press the vessel wall radially outward to increase the diameter of the blood vessel, resulting in increased blood flow. The balloon is then deflated to a small profile so that the dilatation catheter can be withdrawn from the patient's vasculature and the blood flow resumed through the dilated artery. As should be appreciated by those skilled in the art, while the above-described procedure is typical, it is not the only method used in angioplasty.

Another procedure is laser angioplasty which utilizes a laser to ablate the stenosis by super heating and vaporizing the deposited plaque. Atherectomy is yet another method of treating a stenosed body vessel in which cutting blades are rotated to shave the deposited plaque from the arterial wall. A vacuum catheter is usually used to capture the shaved plaque or thrombus from the blood stream during this procedure.

The above non-surgical interventional procedures, when successful, avoid the necessity of major surgical operations. In the procedures of the kind referenced above, however, reclosure may occur or restenosis of the artery may develop over time, which may require another angioplasty procedure, a surgical bypass operation, or some other method of repairing or strengthening the area. Angioplasty or other vascular procedures injure the vessel walls, removing the vascular endothelium, disturbing the tunica intima, and causing the death of medial smooth muscle cells. Excessive neoinitimal tissue formation, characterized by smooth muscle cell migration and proliferation to the intima, follows the injury. Proliferation and migration of smooth muscle cells from the media layer to the intima cause an excessive production of extra cellular matrices, which is believed to be one of the leading contributors to the development of restenosis. The excessive thickening of the tissues narrows the lumen of the blood vessel, constricting or blocking blood flow through the vessel.

To reduce the likelihood of the occurrence of reclosure and to strengthen the area, a physician may implant an intravascular prosthesis for maintaining vascular patency, commonly known as a stent, inside the artery across the lesion. The stent can be crimped onto the balloon portion of the catheter and transported in its delivery diameter through the patient's vasculature. At the deployment site, the stent is expanded to a larger diameter, often by inflating the balloon portion of the catheter.

Therapeutic drug therapy also has been developed over the years to help prevent restenosis. For example, anticoagulant and antiplatelet agents are commonly used to inhibit the development of restenosis. In order to provide an efficacious concentration to the target site, systematic administration of such medication often produces adverse or toxic side effects to the patient. Local treatment may prove to be a preferred method of treatment in that smaller total levels of medication can be administered in comparison to systematic dosage, but are concentrated at a specific site. Local delivery, thus, may produce fewer side effects and possibly achieves more effective results.

Therapeutic drug therapy has been developing in conjunction with stents which are used as the platform for carrying the therapeutic drug or substance to be administered to the damaged vasculature. For example, therapeutic drugs and substances can be impregnated into the body of the stent or carried on a polymeric covering which encases the stent. In this manner, anticoagulants, antiplatelets, and cytostatic substances can be delivered by the stent to prevent thrombosis, to inhibit restenosis and to reduce the growth vascular tissue in the area of treatment. Therapeutic drugs and substances can be released over a period of time once the stent has been implanted in the vasculature.

Drug delivery stents require the therapeutic drug or substance to be absorbed by the body vessel at the site of treatment. However, the amount of therapeutic drug or substance which can be delivered by the stent may be somewhat limited. Also, it is possible for the therapeutic drug or substance to enter the blood stream before it can be fully absorbed by the damaged tissue once the stent is implanted into the patient's vasculature. In order to increase the capacity of the drug delivery stent, the amount of polymeric covering employed, and in effect the thickness of the coating, may be increased to accommodate the quantity of the therapeutic substance used. However, an increase in the profile of the coating may significantly limit the particular application through which the drug delivery stent can be used. Therefore, there is a need to maximize the amount of therapeutic drug or substance to be absorbed by the damaged tissue along with a need to minimize the amount of drug or substance that is swept away in the blood stream.

The amount of therapeutic drug or substance to be introduced at the diseased body vessel and the time needed for the tissue to absorb the substance over a period of time can be regulated to provide more effective drug therapy. In this regard, direct injection of the therapeutic drug or substance into the wall of the body vessel may provide the greatest potency of the drug or substance on the diseased tissue, while also eliminating the drug or substance from being swept away in the blood stream. However, direct injection of the therapeutic drug or substance can be difficult to accomplish in a vascular setting particularly if a syringe is usually utilized to administer the drug. If the drug delivery is to be performed via a non-invasive, percutaneous transluminal procedure, the difficulties in reaching the treatment area are magnified. Therefore, due to the difficulties in reaching and properly injecting the inside wall of the body vessel, the use of localized needle injection drug therapy may not have been fully developed as a viable medical procedure for treating and preventing restenosis or other diseases in a body vessel.

One technique for the local delivery of a therapeutic substance into the tissue surrounding a body vessel is disclosed in U.S. Pat. No. 5,464,395 to Faxon, et al. The Faxon et al. patent discloses a catheter which includes a needle cannula that is slidably disposed within a needle lumen and a balloon which is coupled to the distal end of a catheter. When the balloon is inflated, the needle lumen is brought adjacent to the tissue such that the needle cannula can be moved between a position inboard of the catheter distal surface and a position where the needle cannula is projected out of the catheter to deliver the therapeutic substance to the tissue.

When a balloon is inflated to bring the needle lumen in close engagement with the tissue of the body lumen, the body vessel stretches which may cause some additional disadvantages when piercing the vessel wall with the needle cannula. Generally, the vessel wall should be slightly stretched in order to properly pierce the vessel wall. However, over stretching of the body vessel by a balloon system may make it more difficult to pierce the vessel wall and the change in the vessel wall thickness due to over stretching may cause difficulties in controlling needle penetration depth. Additionally, when a balloon is utilized, the body vessel can become occluded which may prevent vital oxygenated blood from reaching body vessels located downstream from the area of treatment.

The potential release of embolic debris into the bloodstream that can occlude distal vasculature also can cause significant health problems to the patient when a drug delivery stent is implanted in a patient creates another problem. For example, during deployment of a drug delivery stent, it is possible for the metal struts of the stent to cut into the stenosis and shear off pieces of plaque that can travel downstream and lodge somewhere in the patient's vascular system. The same problem exists if drug therapy is to be administered via needle injection. If a drug delivery procedure is to be performed in the carotid arteries, for example, the release of emboli into the circulatory system can be extremely dangerous and may be fatal to the patient. Debris carried by the bloodstream to distal vessels of the brain can cause cerebral vessels to occlude, resulting in a stroke, and in some cases, death. Moreover, large microspheres of the therapeutic substance which may be inadvertently released into the body vessel can cause blockage problems in small vessels located downstream from the treatment area. Therefore, while localized drug therapy can be useful in preventing restenosis, the number of procedures performed, for example, in carotid arteries may be somewhat limited due to the justifiable fear of an embolic stroke.

Filters or traps have been developed in association with angioplasty and stenting procedures for capturing embolic debris before it reaches the smaller blood vessels downstream. The placement of a filter in the patient's vasculature during such treatments can reduce the presence of the embolic debris in the bloodstream. Such embolic filters are usually delivered in a collapsed position through the patient's vasculature and then expanded to trap the embolic debris. Some of these embolic filters are self-expanding and utilize a restraining sheath which maintains the expandable filter in a collapsed position until it is ready to be expanded within the patient's vasculature. The physician can retract the proximal end of the restraining sheath to expose the expandable filter, causing the filter to expand at the desired location. Once the procedure is completed, the filter can be collapsed, and the filter (with the trapped embolic debris) can then be removed from the vessel.

Some prior art expandable filters vessel are attached to the distal end of a guide wire or guide wire-like member which allows the filtering device to be steered in the patient's vasculature as the guide wire is positioned by the physician. Once the guide wire is in proper position in the vasculature, the embolic filter can be deployed to capture embolic debris. The guide wire also can then be used by the physician to deliver interventional devices, such as a balloon angioplasty dilatation catheter or a stent delivery catheter, to perform an interventional procedure in the area of treatment. After the procedure is completed, a recovery sheath can be delivered over the guide wire using over-the-wire techniques to collapse the expanded filter for removal from the patient's vasculature.

What has been needed is a drug delivery system which can be utilized in a patient's vasculature system via a percutaneous transluminal procedure that effectively administers a proper dosage of therapeutic drugs or substance directly into the wall of the patient's body vessel and includes filtering capabilities that allow the system to collect embolic debris or large particles or microspheres of the therapeutic substance that can be released into the patient's vasculature during the drug delivery procedure. Such a system would be advantageous if it eliminates over stretching of the vessel wall during the injection procedure and prevents the blockage of blood flow when deployed. The present invention satisfies these and other needs.

### SUMMARY OF THE INVENTION

The present invention provides an intraluminal substance delivery system having filtering capacity for capturing unwanted substances, such as embolic debris created during the performance of the localized drug delivery or large particles or microspheres of the therapeutic substances which may be released into the bloodstream during the drug delivery procedure. The present invention allows a physician to administer drug therapy to a diseased wall of the patient's vasculature utilizing percutaneous transluminal procedures, such as those performed in coronary angioplasty procedures. The present invention may complement the need for therapeutic drug therapy administered through a drug delivery stent which administers a dosage of therapeutic drug or substance over an extended period of time through bio-absorption. The present invention provides a more direct vehicle for administering a therapeutic drug or substance directly to the diseased wall of a body vessel which may provide greater potency and efficacy of the drug or substance on the diseased tissue. The delivery system of the present invention also performs the drug therapy procedure with a filtering device in place to capture any debris or large particles or microspheres of the therapeutic substance which may be released during the performance of the procedure. As a result, the risk of releasing unwanted debris in critical arteries, such as the carotid arteries, is virtually eliminated since any debris or large microspheres which would otherwise be released into the bloodstream, and possibly induce a stroke, are now captured and collected by the filtering device. The filtering portion of the delivery system also helps to stretch the vessel wall once the filtering portion is deployed within the patient's vasculature. The amount of force exerted by the filtering portion of the system can be controlled in order to prevent over-stretching of the vessel wall.

In one aspect of the present invention, the substance delivery system includes a delivery catheter having one or more delivery lumens coupled with the filtering device. The delivery lumen provides a passageway for the cannula of a conventional delivery syringe or other drug delivery device, such as high pressure jet injectors or needleless injectors, to pass through in order to reach the area to be treated in the patient's vasculature. The delivery lumens can be coupled with the filtering device such that the deployment of the filtering device also deploys and positions the delivery lumens to the location where the drug therapy is to be administered. The delivery catheter also includes a guide wire lumen which receives a guide wire and can be used to position the delivery system at the target area in the patient's vasculature.

In another aspect of the present invention, the filtering device is made from a self-expanding material which allows the device to expand from a low profile, delivery position to an expanded position within the body vessel. In this manner, a protected delivery sheath can be used to co-axially extend over the delivery catheter and filter device to maintain the filter device in the collapsed, low profile delivery position. Once the delivery system is placed in the target area, the delivery sheath can be retracted proximally, which releases the restricting force on the filtering device to allow it to expand within the body vessel. The expansion of the filtering device will, in turn, deploy the delivery lumens near or in direct contact with the diseased wall of the body vessel, allowing the physician to insert the particular drug delivery device into the delivery lumens to the area of treatment.

In another aspect of the present invention, the number of delivery lumens can be increased to provide additional passageways for the delivery of the cannula portion of the drug delivery devices. Additionally, the particular structure of the filter device which forms the embolic filtering device can take on many different forms to achieve the necessary embolic capturing capabilities of the present invention. In another aspect of the invention, the filter device includes an expandable cage having proximal struts coupled to the guide wire and attached to the distal ends of the delivery lumens. These proximal struts move into an expanded position once the delivery sheath is retracted from the filter device, thus allowing the ends of the delivery lumens to be placed near or in contact with the diseased wall of the body vessel. The number of the proximal struts which can be utilized in accordance with the invention can vary depending upon the particular application or particular location of the intended drug therapy.

In another aspect of the present invention, the filtering device can be expanded by mechanical means, such as utilizing a push-pull arrangement in which opposite ends of the expandable cage are pushed inwards to cause the cage to flare outwardly into a deployed position. In this manner, the delivery catheter can be utilized to apply the necessary force on the expandable cage to move the cage into the expanded position. The amount of force applied by the delivery catheter to the expandable cage can be controlled to allow the vessel wall to stretch the necessary amount in order to receive the delivery injection.

It is to be understood that the present invention is not limited by the embodiments described herein. The present invention can be used in arteries, veins, and other body vessels. Other features and advantages of the present invention will become more apparent from the following detailed description of the invention, when taken in conjunction with the accompanying exemplary drawings.

### BRIEF DESCRIPTION OF THE DRAWINGS

FIG. 1 is a perspective view of an intraluminal substance delivery system with an embolic filtering capability embodying features of the present invention.

FIG. 2 is an elevational view, partially in cross section, of the intraluminal substance delivery system with embolic filtering capability of FIG. 1 being delivered within a body vessel to an area to be treated with localized drug therapy.

FIG. 3 is an elevational view, partially in cross section, similar to that shown in FIG. 2, wherein the intraluminal substance delivery system with embolic filtering capability has been deployed with the embolic filtering device placed in its expanded position and the ends of the drug delivery syringes inserted into the diseased wall of the body vessel.

FIG. 4 is a cross-sectional view of the delivery system taken along line 4-4.

FIG. 5 is a cross-sectional view of the delivery system taken along line 5-5.

FIG. 6 is a perspective view of the expandable cage which forms part of the filtering device depicted in FIGS. 1-3.

FIG. 7 is a perspective view of another embodiment of an intraluminal substance delivery system with filtering capability which embodies features of the present invention.

FIG. 8 is a perspective view of still another embodiment of an intraluminal substance delivery system with filtering capability which embodies features of the present invention.

FIG. 8A is a cross-sectional view of the delivery catheter taken along line 8A-8A.

FIG. 9 is an elevational view, partially in cross section, of the intraluminal substance delivery system of FIG. 8 as it has been deployed with the embolic filtering device placed in its expanded position and the ends of drug delivery devices inserted into the diseased wall of the body vessel.

FIG. 10 is an elevational view showing one way to connect the delivery lumen of the delivery catheter to a proximal strut of the expandable cage forming the filtering device.

FIG. 11 is an elevational view showing one way to connect the delivery lumen of the embodiment of FIG. 7 to the expandable cage of the filtering device.

FIG. 12 is an elevational view showing another way to connect the delivery lumen of the embodiment of FIG. 7 to the expandable cage of the filtering device.

### DETAILED DESCRIPTION OF THE PREFERRED EMBODIMENTS

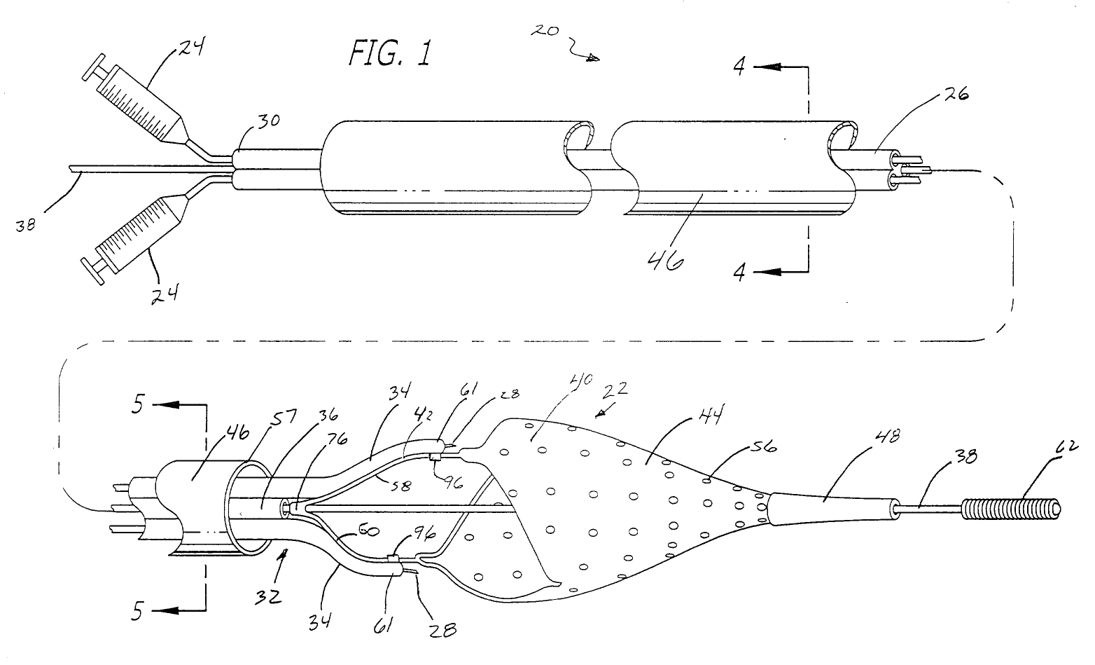

Turning now to the drawings, in which like reference numerals represent like or corresponding elements in the drawings, FIG. 1 illustrates one particular embodiment of an intraluminal substance delivery system 20 including a filtering device 22 incorporating features of the present invention. The delivery system 20 of the present invention is used to administer a therapeutic drug or substance intraluminally in a patient's vasculature via direct drug injection. The filtering device 22 is, in turn, designed to capture embolic debris which may be created and released into a body vessel during the drug delivery procedure, or large particles or microspheres of the therapeutic substance that may be released during the injection procedure. The filtering device 22 is adapted to help position the drug delivery device, here shown as a hypodermic syringe 24, near or in contact with the wall of the body vessel at the location where the therapeutic drug or substance is to be administered. Although the delivery system 20 is described herein in use with a hypodermic syringe, which is one type of drug delivery device that can be used with the system, it should be appreciated that other drug delivery devices, such as high pressure jet injection devices and needleless injectors, also could be used to administer the drug therapy. Also, it should also be appreciated that the delivery system 20 could be used for placement of other medical devices, such as pressure sensors, at a remote location within the patient's vasculature as well. As such, the delivery system of the present invention can be used with both drug delivery devices and other medical devices which may require intraluminal placement in remote locations within a patient.

As can be seen in FIG. 1, the delivery system 20 includes a delivery catheter 26 having a pair of delivery lumens used to facilitate the placement of the ends 28 of the syringes 24 within the patient's vasculature. The delivery catheter 26 has proximal end 30 extending outside of the patient which can be manipulated by the physician when performing the drug delivery procedure using percutaneous transluminal procedures known in the art. The distal end 32 of the delivery catheter 26 terminates at the filtering device 22 which helps to position the ends 28 of the syringes 24 near or in contact with the vascular wall (FIG. 3) to facilitate the administration of the therapeutic drug or substance to the patient. In the particular embodiment shown and described herein, the distal end 32 of the delivery catheter 26 includes a pair of delivery lumens 34 and a separate guide wire lumen 36 which receives the guide wire 38 used to deliver the delivery system 20 within the patient.

The filtering device 22 includes an expandable filter assembly 40 having an expandable basket or cage 42 and a filter element 44 attached thereto. This expandable basket can be made from a self-expanding material, such as nickel-titanium alloy (nitinol), or it may be opened and closed by mechanical means. In this particular embodiment, the expandable filter assembly 40 has self-expanding properties and is mounted on the distal end of an elongated (solid or hollow) cylindrical tubular shaft, such as a steerable guide wire 38. The guide wire 38 also has a proximal end which extends outside of the patient that can be manipulated by the physician to deliver the system 20 into the target area in the patient's vasculature. A restraining or delivery sheath 46 extends coaxially along the guide wire 28 in order to maintain the expandable filter assembly 40 in its collapsed position until it is ready to be deployed within the patient's vasculature. The delivery sheath also creates a protective covering over the delivery catheter and filtering assembly as these components move within the patient's vasculature. The expandable filter assembly 40 can be deployed by the physician by simply retracting the restraining sheath 46 proximally to expose the expandable filter assembly 40. Once the restraining sheath 46 is retracted, the self-expanding cage 42 immediately begins to expand within the body vessel (see FIG. 3), causing the filter element 44 to expand and contact the vessel wall, while positioning the delivery lumens near or in contact with the wall.

An obturator 48 affixed to the distal end of the filter assembly 40 can be implemented to prevent possible “snowplowing” of the drug delivery system 20 as it is being delivered through the vasculature. The obturator can be made from a soft polymeric material, such as Pebax 40D, and has a smooth surface to help the embolic filtering device travel through the vasculature and cross lesions while preventing the distal end of the restraining sheath 46 from otherwise “digging” or “snowplowing” into the walls of the vasculature.

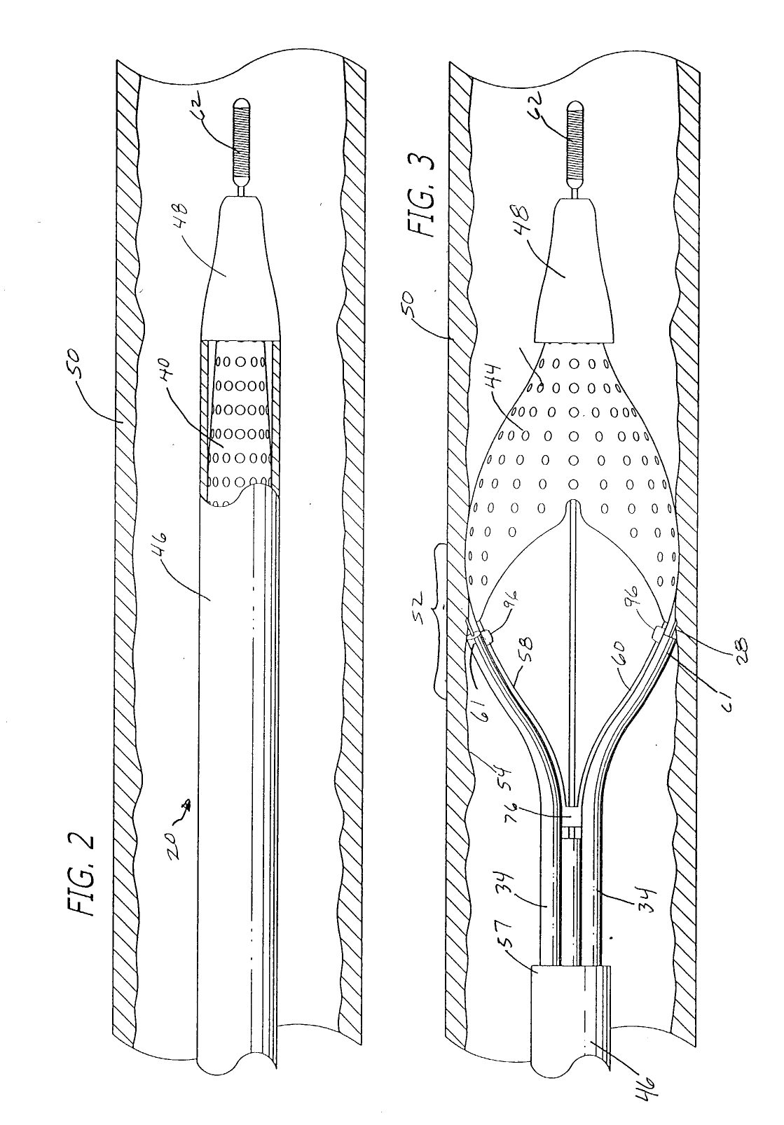

In FIGS. 2 and 3, the delivery system 20 is shown as it is being delivered within an artery 50 or other body vessel of the patient. The system 20 made in accordance with the present invention should have sufficient bendability and flexibility for ease of delivery, as well as being conformable to the shape of the vasculature, to allow the system to negotiate a curved radius in the patient's vasculature. Referring now to FIG. 3, the delivery catheter 26 and the filtering device 22 are shown in an expanded position within the patient's artery 50. This portion of the artery (FIG. 3) has an area of treatment 52 in which a therapeutic drug or substance is to be administered in the diseased wall 54 of the artery 50. The area of treatment is typically a diseased portion of the body vessel where, for example, atherosclerotic plaque has built up against the wall of the artery and which was previously treated, for example, with a balloon angioplasty procedure or stenting procedure to enlarge the vessel diameter. However, the use of the drug delivery system 20 made in a accordance with the present invention is not limited to the treatment of atherosclerotic plaque, but can be used to treat any disease which afflicts the vasculature of a patient. It also should be appreciated that the embodiments of the delivery systems and embolic filtering device described herein are illustrated and described by way of example only and not by way of limitation. Also, while the present invention is described as applied generally to an artery of the patient, those skilled in the art will appreciate that it can be used in the coronary arteries, carotid arteries, renal arteries, saphenous vein grafts, other peripheral arteries and body vessels.

The expandable cage 42 includes self-expanding struts which, upon release from the restraining sheath 46, expand the filter element 44 into its deployed position within the artery (FIG. 3). Embolic particles (not shown) created during the drug delivery procedure and released into the bloodstream, or large microspheres of the therapeutic substance can be captured within the deployed filter element 44. The filter includes perfusion openings 56, or other suitable perfusion means, to allow blood flow through the filter element 44. The filter element will capture embolic particles which are larger than the perfusion openings while allowing some blood to perfuse downstream to vital organs.

Referring specifically to FIGS. 1, 3 and 6, the expandable cage 42 includes two self-expanding proximal struts 58 and 60 which help to deploy the filtering element 44 and place the ends 61 of the delivery lumens 34 near or in contact with the wall of the artery. In use, the delivery system is advanced to the desired location of the artery where the therapeutic drug or substance is to be injected into the wall of the artery. The delivery of the delivery system 20 is performed by the physician through manipulation of the guide wire 38 and its coil tip 62 using techniques well known in the art. Thereafter, once in position, the delivery sheath 46 can be retracted proximally by the physician to move the restraining force placed on the expandable cage 42, thus allowing the filter assembly 40 to expand within the artery. In this manner, the ends 61 of the delivery lumens 34 are attached to the proximal struts 58 and 60 move into position near or against the diseased wall 54 of the artery 50. Once the filter assembly 40 is in proper position, the physician can then place the cannulas 63 of the syringes 24 into the proximal openings of the syringe lumens 34 to advance the ends 28 of the syringes along the delivery catheter in order to pierce the wall 54 of the artery 50. The physician can then administer the desired dosage of therapeutic drug or substance from the proximal end of the delivery syringes.

After the physician injects the proper drug dosage to the area of treatment 52, the physician can then advance the sheath 46 distally to collapse the filter assembly 40 back into its collapsed, delivery configuration. This action occurs when the distal end 57 of the sheath 46 contacts the delivery lumens 34 and proximal struts 58 and 60 to impart a retracting force on the expandable cage 42 causing the cage to move back to a collapsed position. The filter assembly 40 should contain any debris which may have been created during the administration of the therapeutic substance into the wall of the artery. The embolic particles will remain within the filter element 44 as the filter assembly 40 is being collapsed via the action of the sheath 46 on the assembly. Thereafter, the physician can move the system to yet another area to be treated by therapeutic drugs or substances. Alternatively, once the drug or substance is injected to the patient, the physician has the option of simply removing the delivery system from the vasculature. It should be appreciated that other procedures can be performed by the physician which deviates somewhat from the specific steps discussed above without departing from the spirit and scope of the present invention.

The present invention can be made in accordance with an over-the-wire delivery system in which the guide wire is first placed into the desired area of the vasculature. The drug delivery system 20 with filtering device 22 can be advanced over the guide wire in an over-the-wire fashion using techniques well known in the art to position the distal end of the system at the desired location. In this manner, the guide wire 38 would be longitudinally moveable within the guide wire lumen 36 of the delivery catheter to allow the entire delivery system 20 to move along the length of the guide wire. Alternatively, the guide wire can be permanently attached to the delivery system to create a composite system which is advanced together with the guide wire into the desired location of the patient's vasculature.

The filter assembly 40 shown in FIGS. 1-4 can be easily adapted to a push-pull system in which the need for a self-expanding cage 42 is eliminated. In this particular embodiment, the expandable cage 42 would be open by simply applying force to the distal and proximal end of the expandable cage 42 to cause it to spring open. Spring steel could be used to manufacture this type of expandable cage. In such an embodiment, one end of the cage would be attached, for example, to the guide wire with the delivery catheter 42 being used to apply force to the proximal end of the cage 42. In this manner, once the expandable cage reaches the area to be treated, application of force to the expandable cage will force it to expand, bringing the delivery lumens in close proximity to the wall of the body vessel. A fitting (not shown) located near the distal end of the guide wire could be used to contact the expandable cage to provide abutting shoulder to hold the cage as force is being applied to the cage via the delivery catheter. Alternatively, the distal portion of the expandable cage could be permanently attached to the guide wire. The entire guide wire and delivery catheter could be advanced together within the patient's vasculature for deployment. The use of a fitting on the guide wire, however, allows the delivery system and filter to be delivered over the guide wire once the guide wire has been initially positioned in the patient.

Referring specifically to FIGS. 1 and 6, the particular structure of the filter assembly 40 can be seen. The proximal struts 58 and 60 are coupled to a circumferential member 66 which is adapted to move from the unexpanded, delivery position (FIG. 2) to the expanded, deployed position (FIG. 3). A pair of distal struts 68 and 70 are connected to the circumferential member 66 and extend distally towards the obturator 48.

As can be seen best in FIG. 6, the circumferential member 66 is formed in a zig-zag pattern which includes apexes to which the proximal and distal struts are attached. These apexes form bending regions 72 which enhance the ability of the circumferential member to bend as it moves between the unexpanded and expanded positions. In the particular embodiment shown in FIG. 6, each bending region 72 is placed on the circumferential member approximately 90 degrees apart. Each of the proximal struts includes a first end 74 attached to a collar 76 that is mounted to the guide wire 38. Alternatively, the proximal struts may be attached directly to the guide wire. Each proximal strut includes a second end 78 connected to one of the bending regions 72 of the circumferential member 66. Likewise, each of the distal struts 68 and 70 includes a first end 80 connected to, and extending towards, the obturator 48 and a second end 82 attached to distally located bending regions 72 on circumferential member 66.

Each of the bending regions is substantially U-shaped which help to create a natural bending point on the circumferential member. While the flexibility of the circumferential members is already high, these bending regions help to increase the ability of the circumferential member to collapse or expand when needed. In this manner, the shape of the hinge regions creates a natural hinge that helps to actuate the expandable cage between the unexpanded and expanded positions. As can be best seen in FIG. 6, the U-shaped proximally located bending regions are positioned directly opposite the U-shaped portion of the distally located bending regions. The positioning of the direction of the U portion also enhances the ability of the circumferential member to bend. The circumferential member, while being quite bendable, nevertheless maintains sufficient radial strength to remain in the deployed position to maintain the delivery lumens 34 near or in contact with the wall of the body vessel and to hold the filter element 44 in position for collecting embolic particles which may be entrained in the body fluid.

It should be appreciated that the expandable cage 42 shown in FIGS. 1 and 6 is just one of a number of many cage designs which can be utilized to provide filtering capability to the substance delivery system. Moreover, the filtering assembly itself does not have to be self-expanding, as is disclosed in the embodiment of FIGS. 1 and 6, but rather, can be collapsed and expanded by any one of a number of different mechanisms. For example, the expandable cage can be moved between its unexpanded position and expanded position utilizing a push-pull mechanism in which the opposite ends of the cage are pushed inward to cause the struts to flare outward into a deployed position. Expandable filters and the mechanisms used to impart such a flared out deployment are known in the art and are disclosed, for example, in U.S. Pat. No. 5,989,281 and U.S. Pat. No. 6,027,520. Other mechanisms for deploying the struts of the cage include fluid expandable lumens and balloon-like members which can be inflated to cause the struts of the cage to expand radially outward are shown in U.S. Pat. Nos. 6,011,118 and 5,827,324. These are just some of the many examples of mechanisms that can be utilized to deploy the embolic filtering apparatus and delivery lumens of the present invention.

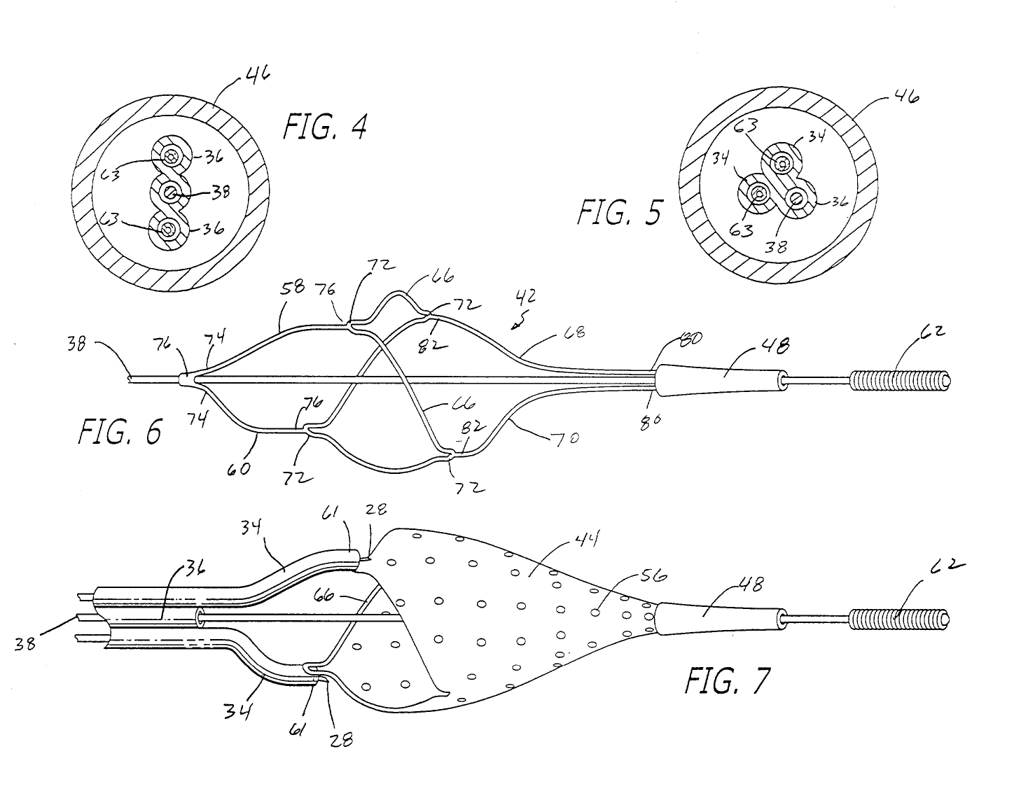

The configuration of the delivery catheter 26 can take on various shapes as well. For example, as is shown in FIGS. 1, 4 and 5, the structure and location of the various lumens which form the delivery catheter can vary. For example, as is shown in FIGS. 1 and 4, the distal ends of the delivery catheter 26 can be formed in a side-by-side arrangement in which the guide wire lumen 36 is disposed between the pair of delivery lumens 34. This allows the delivery lumens 34 to be maintained at an outer locations to facilitate attachment to the proximal struts and to prevent the delivery lumens from crossing each other. This particular arrangements helps to facilitate deployment of the delivery lumens 34 in connection with the filtering assembly 40. The delivery catheter can take on a different profile at different locations on the catheter. For example, as is shown in FIGS. 1 and 5, the guide wire lumen 36 and delivery lumens 34 can be placed closer together along the main length of catheter to create a smaller profile which has sufficient rigidity and pushability where it is needed, namely, along the major shaft of the delivery catheter. Accordingly, the particular arrangement of guide wire and delivery lumens can take on many different forms and shapes without departing from the scope of the present invention. Moreover, particular portions of the delivery catheter can be made from different materials, as may be needed, in order to accommodate the particular flexibility or strength that is needed for that particular position. Those skilled in the art will recognize that the major shaft portion of the delivery catheter can be made with more rigid materials since additional pushability and strength is usually needed for this portion of the catheter. In this manner, the distal most end of the delivery catheter may be made with more flexible and thinner wall delivery lumens to provide the flexibility needed in order to reach the sometimes tortuous areas into which the system will be advanced. Additionally, the number of delivery lumens can be varied as needed for a particular therapy. While the embodiments of FIGS. 1-6 utilize a pair of delivery lumens 34, it should be appreciated that a single delivery lumen could be utilized without departing from the scope of the present invention.

Substance delivery devices, such as syringes 24 used with the present invention accordingly have large cannula lengths which must be flexible as well. In this regard, the cannula of the syringe made be made from a material such as nickel-titanium alloy hypotubing, which has sufficient flexibility and bendability to allow the physician to advance the cannula into the lumens to reach the distal most end where the therapeutic drug or substance is to be administered. The proximal end of the syringe cannula may include a stop fitting (not shown) which abuts against the proximal end of the delivery catheter to prevent further advancement of the end of the cannula past a certain measured distance. In this fashion, the physician can be assured that only the needed length of the syringe cannula extends outwardly from the delivery lumen. It should be appreciated that if the end of the syringe cannula extends too far out from the delivery lumen, it is possible for the end to pass completely through the vessel wall, is not desired. In this manner, the stop fitting will allow only the desired length of the syringe to extend outside of the delivery lumen. It should also be appreciated that other means for regulating the precise length of cannula that can extend out of the lumen also could be implemented. Accordingly, if drug or substance delivery devices such as high pressure jet injectors or needleless injectors are utilized with the delivery system, it should be appreciated that the cannula used with these devices do not have to penetrate the vessel wall, but rather, can be placed in close proximity to the wall to allow the drug to be injected into the tissue. In this regard, the end of the cannula used in accordance with such devices also could be regulated with stop fittings or other mechanical means to properly position the cannula adjacent to the area to be treated.

Referring now to FIG. 7, an alternative embodiment of the filtering assembly 40 is shown. In this particular embodiment, the expandable cage 42 lacks the proximal struts such as those found on the cage shown in FIG. 6. Rather, the delivery lumens 34 are directly attached to the circumferential member 66 and move with the circumferential member 66 as it expands within the body vessel. This particular embodiment of the filter assembly would be moved to the collapsed position through the use of the sheath 46. The action of the distal end of the sheath on the delivery lumens 34 and filter assembly 46 could be the same as described above. The sheath would be advanced distally to contact the delivery lumens 34 to cause the delivery lumens 34 and circumferential member 66 to move back into the collapsed position.

The particular expandable cage 42 utilized in the embodiment of shown in FIG. 7 is sometimes referred to as a half-basket or half-cage embodiment. In this regard, the materials forming the circumferential member is typically self-expanding to allow the filter assembly to move into its expanded position once the restraining force imparted by the delivery sheath is removed. The delivery lumens 34 can be attached to the circumferential member 66 in a variety of ways. For example, as shown in FIGS. 11 and 12, the end 84 of the delivery lumen 34 can be bonded or adhesively attached to the circumferential member utilizing an adhesive-type material 86 which affixes the elements together. Suitable bonding materials are well-known in the field. Alternatively, as is shown in FIG. 12, the end 84 of the delivery lumen 34 can be attached to the circumferential member 66 utilizing a band 88, or a similar fastening mechanism which holds the end 84 directly to the circumferential member 66. Other ways of attaching the ends of the delivery lumen to the filter assembly can be accomplished. The exact positioning of the delivery lumens with respect to the filter assembly will depend upon the type of filtering assembly utilized in conjunction with the present invention.

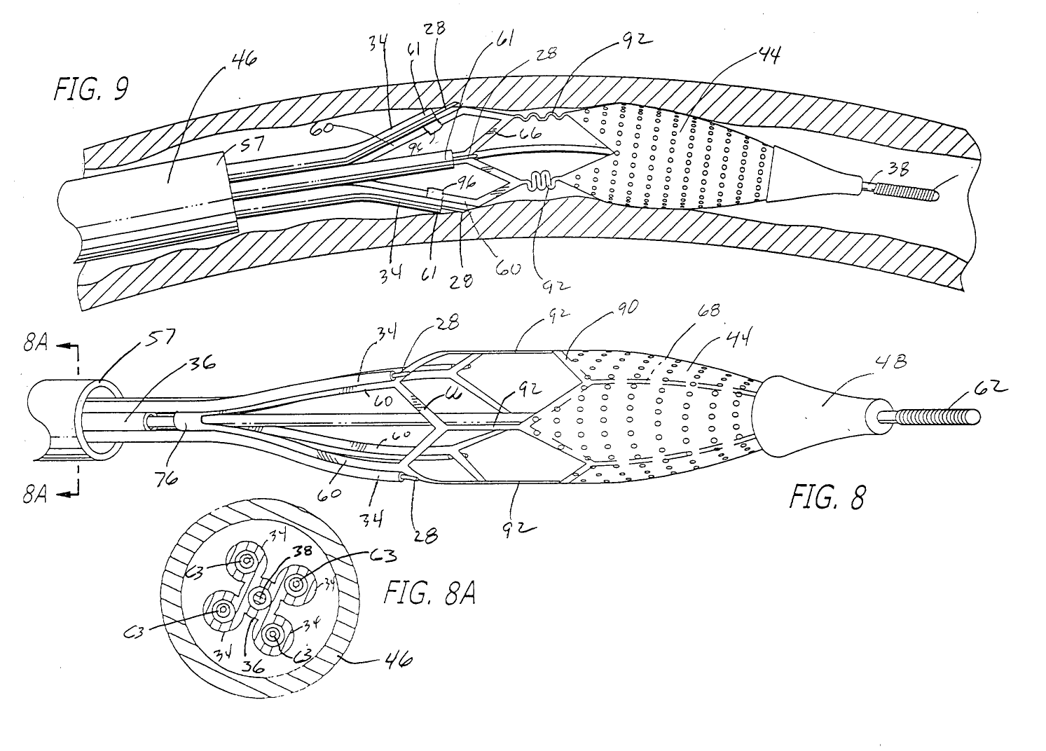

Referring now to FIGS. 8, 8A and 9, an alternative embodiment of the delivery system 20 is shown. As can be seen more clearly in FIG. 9, the particular filtering device 90 is somewhat different from the previously disclosed embodiments in that the filter element 44 is located more distally from the delivery lumens 34. This allows the area of treatment to be located more upstream from the deployed filter element 44 during use. This configuration may provide a better means for capturing embolic debris which may be created during the substance delivery procedure.

Referring specifically now to FIG. 8, another particular cage design for the filter assembly 40 is shown. The cage 42 includes four proximal struts 60 attached to a first circumferential member 66, similar to the previously described embodiment shown in FIGS. 1-3. The cage 42 is longer in length since a second circumferential member 90 is connected via interconnecting links 92 to form the composite cage. The interconnecting links 92 may be linear or non-linear in shape which will allow the cage to bend a bit more easily when deployed in a curved portion of the body vessel, such as that shown in FIG. 9. The second circumferential member includes four distal struts 68 which extend from the circumferential member to the end of the cage which is covered by the obturator 48. The filter element 44 is attached to this second circumferential member 90.

In this particular embodiment, four proximal struts 60 provide the means for deploying four individual delivery lumens 34 which form part of the delivery catheter 26. As can be seen in FIG. 8A, four individual delivery lumens 34 are located around the guide wire lumen 36 which can be located in the center of the delivery catheter to allow the delivery lumens 34 to extend outward and attach to the proximal struts 60. This particular embodiment of the delivery system 20 would be used in substantially the same manner and procedure as described above except now the physician has four separate delivery lumens which he/she can utilize while administering the therapeutic drug or substance via the syringes. Those skilled in the art will again recognize that the number of proximal struts, along with the number of delivery lumens which can be utilized in accordance with the present invention can vary, and that designs could be implemented in which there are more proximal struts than delivery lumens and visa-versa. Again, it should be appreciated that these embodiments show some of the numerous configurations of embolic protection assemblies that can be utilized in accordance with delivery catheters to provide a composite drug delivery system made in accordance with the present invention.

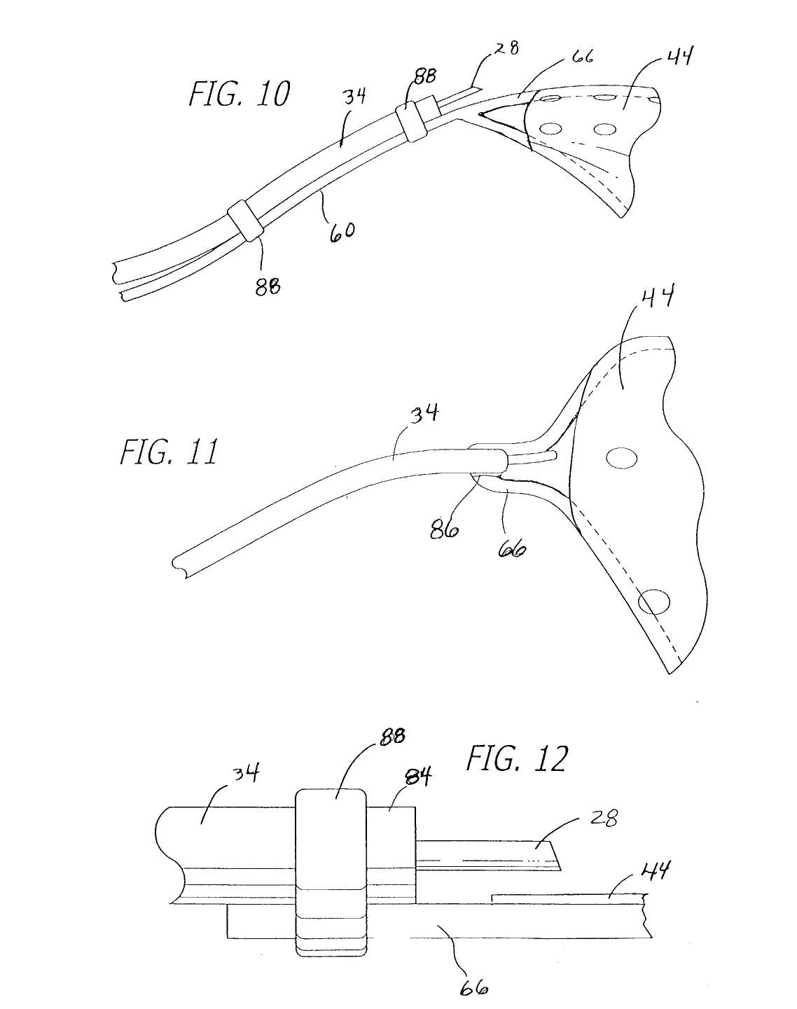

Referring now to FIG. 10, one way in which the delivery lumens 34 can be attached to the proximal struts 60 is shown. In this particular arrangement, one or more attachment bands 88, or other similar attachment member, can be utilized to attach the proximal strut. It should also be appreciate that adhesives and other attachment mechanisms could be utilized as well.

The delivery system may also include strategically placed radiopaque markers 96 which enhance visualization during flouroscopy. For example, the radiopaque markers 96 can be placed near the ends of the delivery lumens to allow the physician to visualize the positioning of the drug delivery device near the body vessel wall. Still other radiopaque markers could also be utilized in accordance with the delivery system and filtering device in order to enhance visualization.

It should also be appreciated that the particular angle at which the delivery lumens are placed on any of the filter assembly disclosed therein can be varied depending upon the structure of the assembly. In this manner, the angle at which the end of the syringe enters the wall of the body vessel can be varied as needed. For example, the angle of entry of the needle cannula can be adjusted by adjusting the angle of expansion of the struts of the cage when deployed into the expanded position.

The therapeutic drugs or substances that can be delivered via the drug delivery system include, but are not limited to, antineoplastic, antimitotic, anti-inflammatory, antiplatelet, anticoagulant, antifibrin, antithrombin, antiproliferative, antibiotic, antioxidant, and antiallergenic substances as well as combinations thereof. Examples of such antineoplastics and/or antimitotics includes paclitaxel (e.g., TAXOL® by Bristol-Meyers Squibb Co., Stamford, Conn.), docetaxel (e.g., Taxotere® from Aventis S.A., Frankfurt, Germany), methotrexate, azathioprine, vincristine, vinblastine, fluorouracil, doxorubicin hydrochloride (e.g., Adriamycin® from Pharmacia & Upjohn, Peapack, N.J.), and mitomycin (e.g., Mutamycin® from Bristol-Meyers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as Angiomax™ (Biogen, Inc., Cambridge, Mass.). Examples of such cytostatic or antiproliferative agents include angiopeptin, angiotensin converting enzyme inhibitors such as captopril (e.g., Capoten® and Capozide® from Bristol-Meyers Squibb Co., Stamford, Conn.), cilazapril or lisinopril (e.g., Prinivil® and Prinzide® from Merck & Co., Inc., Whitehouse Station, N.J.); calcium channel blockers (such as nifedipine), colchicine, fibroblast growth factor (FGF) antagonists, fish oil (omega 3-fatty acid), histamine antagonists, Lovastatin® (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phophodiesterase inhibitors, prostaglandin inhibitors, suramin, seratonin blockers, steroids, thioprotease inhibitors, triazolpyrimidine (a PDGF antagonist), and nitric oxide. Antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as Angiomax™ (Biogen, Inc., Cambridge, Mass.). An example of an antiallergenic agent is permirolast potassium. Other therapeutic substances or agents that may be used include alpha-interferon, genetically engineered epithelial cells, and dexamethasone. In other examples, the therapeutic substance may be a radioactive isotope for prosthesis usage in radiotherapeutic procedures. Examples of radioactive isotopes include, but are not limited to, phosphoric acid (H₃P³²O₄), palladium (Pd¹⁰³), cesium (Cs¹³¹), and iodine (I¹²⁵). While the preventative and treatment properties of the foregoing therapeutic substances or agents are well-known to those of ordinary skill in the art, the substances or agents are provided by way of example and are not meant to be limiting. Other therapeutic drug or substances which are currently available or that may be developed are equally applicable for use with the present invention. Additionally, other substances which can be administered include angiogenesis agents, including agents used in cell therapy and gene therapy.

The expandable cage of the present invention can be made in many ways. One particular method of making the cage is to cut a thin-walled tubular member, such as nickel-titanium hypotube, to remove portions of the tubing in the desired pattern for each strut, leaving relatively untouched the portions of the tubing which form the structure. The tubing may be cut into the desired pattern by means of a machine-controlled laser. The tubing used to make the cage could possible be made of suitable biocompatible material, such as spring steel. Elgiloy is another material which could possibly be used to manufacture the cage. Also, very elastic polymers possibly could be used to manufacture the cage. Spring steel is a suitable material for a cage which is force actuated.

The strut size is often very small, so the tubing from which the cage is made may have a small diameter. Typically, the tubing has an outer diameter on the order of about 0.020-0.040 inches in the unexpanded condition. Also, the cage can be cut from large diameter tubing. Fittings are attached to both ends of the lased tube to form the final cage geometry. The wall thickness of the tubing is usually about 0.076 mm (0.001-0.010 inches). As can be appreciated, the strut width and/or depth at the bending points will be less. For cages deployed in body lumens, such as PTA applications, the dimensions of the tubing may be correspondingly larger. While it is preferred that the cage be made from laser cut tubing, those skilled in the art will realize that the cage can be laser cut from a flat sheet and then rolled up in a cylindrical configuration with the longitudinal edges welded to form a cylindrical member.

Generally, the tubing is put in a rotatable collet fixture of a machine-controlled apparatus for positioning the tubing relative to a laser. According to machine-encoded instructions, the tubing is then rotated and moved longitudinally relative to the laser which is also machine-controlled. The laser selectively removes the material from the tubing by ablation and a pattern is cut into the tube. The tube is therefore cut into the discrete pattern of the finished struts. The cage can be laser cut much like a stent is laser cut. Details on how the tubing can be cut by a laser are found in U.S. Pat. Nos. 5,759,192 (Saunders), 5,780,807 (Saunders) and 6,131,266 (Saunders) which have been assigned to Advanced Cardiovascular Systems, Inc.

The process of cutting a pattern for the strut assembly into the tubing generally is automated except for loading and unloading the length of tubing. For example, a pattern can be cut in tubing using a CNC-opposing collet fixture for axial rotation of the length of tubing, in conjunction with CNC X/Y table to move the length of tubing axially relative to a machine-controlled laser as described. The entire space between collets can be patterned using the CO₂ or Nd:YAG laser set-up. The program for control of the apparatus is dependent on the particular configuration used and the pattern to be ablated in the coding.

A suitable composition of nickel-titanium which can be used to manufacture the strut assembly of the present invention is approximately 55% nickel and 45% titanium (by weight) with trace amounts of other elements making up about 0.5% of the composition. The austenite transformation temperature is between about 0° C. and 20° C. in order to achieve superelasticity at human body temperature. The austenite temperature is measured by the bend and free recovery tangent method. The upper plateau strength is about a minimum of 60,000 psi with an ultimate tensile strength of a minimum of about 155,000 psi. The permanent set (after applying 8% strain and unloading), is less than approximately 0.5%. The breaking elongation is a minimum of 10%. It should be appreciated that other compositions of nickel-titanium can be utilized, as can other self-expanding alloys, to obtain the same features of a self-expanding cage made in accordance with the present invention.

In one example, the cage of the present invention can be laser cut from a tube of nickel-titanium (Nitinol) whose transformation temperature is below body temperature. After the strut pattern is cut into the hypotube, the tubing is expanded and heat treated to be stable at the desired final diameter. The heat treatment also controls the transformation temperature of the cage such that it is super elastic at body temperature. The transformation temperature is at or below body temperature so that the cage is superelastic at body temperature. The cage is usually implanted into the target vessel which is smaller than the diameter of the cage in the expanded position so that the struts of the cage apply a force to the vessel wall to maintain the cage in its expanded position. It should be appreciated that the cage can be made from either superelastic, stress-induced martensite NiTi or shape-memory NiTi.

The cage could also be manufactured by laser cutting a large diameter tubing of nickel-titanium which would create the cage in its expanded position. Thereafter, the formed cage could be placed in its unexpanded position by backloading the cage into a restraining sheath which will keep the device in the unexpanded position until it is ready for use. If the cage is formed in this manner, there would be no need to heat treat the tubing to achieve the final desired diameter. This process of forming the cage could be implemented when using superelastic or linear-elastic nickel-titanium.

The struts forming the proximal struts can be made from the same or a different material than the distal struts. In this manner, more or less flexibility for the proximal struts can be obtained. When a different material is utilized for the struts of the proximal struts, the distal struts can be manufactured through the lazing process described above with the proximal struts being formed separately and attached. Suitable fastening means such as adhesive bonding, brazing, soldering, welding and the like can be utilized in order to connect the struts to the distal assembly. Suitable materials for the struts include superelastic materials, such as nickel-titanium, spring steel, Elgiloy, along with polymeric materials which are sufficiently flexible and bendable.

The polymeric material which can be utilized to create the filtering element include, but is not limited to, polyurethane and Gortex, a commercially available material. Other possible suitable materials include ePTFE. The material can be elastic or non-elastic. The wall thickness of the filtering element can be about 0.00050-0.0050 inches. The wall thickness may vary depending on the particular material selected. The material can be made into a cone or similarly sized shape utilizing blow-mold technology or dip molding technology. The openings can be any different shape or size. A laser, a heated rod or other process can be utilized to create to perfusion openings in the filter material. The holes, would of course be properly sized to catch the particular size of embolic debris or microspheres of interest. Holes can be lazed in a spinal pattern with some similar pattern which will aid in the re-wrapping of the media during closure of the device. Additionally, the filter material can have a “set” put in it much like the “set” used in dilatation balloons to make the filter element re-wrap more easily when placed in the collapsed position.

The materials which can be utilized for the restraining sheath and delivery catheter can be made from polymeric materials, such as cross-linked HDPE. The delivery catheter can be made using known polymeric materials that are used for angioplasty balloon catheters and similar medical devices. The sheath can alternatively be made from a material such as polyolifin which has sufficient strength to hold the compressed strut assembly and has relatively low frictional characteristics to minimize any friction between the filtering assembly and the sheath. Friction can be further reduced by applying a coat of silicone lubricant, such as Microglide®, to the inside surface of the restraining sheath before the sheaths are placed over the filtering assembly.

Further modifications and improvements may additionally be made to the device and method disclosed herein without departing from the scope of the present invention. Accordingly, it is not intended that the invention be limited, except as by the appended claims.

What is claimed is:

1. A delivery system for administering therapeutic drugs or substances in a body vessel of a patient, comprising:
a delivery catheter having a proximal end and a distal end and a delivery lumen for receiving the cannula of a syringe, the delivery lumen having a distal end through which the cannula extends for the administration of a therapeutic drug or substance to the body vessel; and
means associated with the delivery catheter for capturing unwanted particles which may be entrained in the body fluid passing through the body vessel.

2. The delivery system of claim 1, further including a delivery sheath adapted to extend co-axially over the delivery catheter and the particle capturing means when the delivery system is being advanced within the body vessels of the patient.

3. The delivery system of claim 1, wherein the particle capturing means includes means for deployment in the body vessel.

4. The delivery system of claim 3, wherein the distal end of the delivery lumen is coupled to the particle capturing means and is positioned within the body vessel when the particle capturing means is deployed.

5. The delivery system of claim 4, wherein the particle filtering means is an embolic filtering device having an expandable cage and a filter element attached to the expandable cage, the distal end of the delivery lumen being attached to the filtering device.

6. The delivery system of claim 5, wherein the expandable cage of the filtering device is self-expanding.

7. The delivery system of claim 6, further including a delivery sheath adapted to extend co-axially over the delivery catheter and the filtering device when the drug delivery system is being advanced within the body vessels of the patient.

8. The delivery system of claim 7, wherein the delivery sheath is adapted to extend over the filtering device to maintain a restraining force on the filtering device which is removable once the delivery sheath is retracted from the filtering device.

9. The delivery system of claim 1, wherein the delivery catheter includes a plurality of delivery lumens.

10. The delivery system of claim 1, wherein the particle capturing means is placed at a distal location from the distal end of the delivery lumen.

11. The delivery system of claim 5, wherein the expandable cage includes a proximal end and a distal end and the expandable cage is expanded through application of a force to one or both of the distal and proximal ends of the expandable cage.

12. A delivery system for administering therapeutic drugs or substances in a body vessel of a patient, comprising:
a delivery catheter having a proximal end and a distal end and a delivery lumen for receiving the cannula of a syringe, the delivery lumen having a distal end through which the cannula extends for the administration of a therapeutic drug or substance to the body vessel; and
a filtering device associated with the delivery catheter for capturing unwanted particles which may be entrained in the body fluid passing through the body vessel.

13. The delivery system of claim 12, wherein the positioning of the end of the delivery lumen is accomplished when the filtering device is deployed in the body vessel.

14. The delivery system of claim 12, wherein the filtering device is movable between an unexpanded position and an expanded position and the distal end delivery lumen is attached to the filtering device.

15. The delivery system of claim 12, wherein the filtering device includes an expandable cage and a filter element attached thereto.

16. The delivery system of claim 12, wherein the expandable cage is made from a self-expanding material.

17. The delivery system of claim 15, wherein the expandable cage includes a proximal end and a distal end and the expandable cage is expanded through application of a force to one or both of the distal and proximal ends of the expandable cage.

18. The delivery system of claim 12, further including a delivery sheath adapted to extend over the filtering device to maintain a restraining force on the filtering device which is removable once the delivery sheath is retracted from the filtering device.

19. The delivery system of claim 12, further including a delivery sheath adapted to extend co-axially over the delivery catheter and the filtering device when the delivery system is being advanced within the body vessels of the patient.

20. The delivery system of claim 15, wherein the expandable cage includes a proximal strut which extends outwardly in a radial fashion when placed in the expanded position, the end of the delivery lumen being attached thereto.

21. The delivery system of claim 12, further including a plurality of delivery lumens.

22. The delivery system of claim 12, wherein the delivery catheter has a plurality of delivery lumens and a guide wire lumen for receiving a guide wire.

23. The delivery system of claim 22, further including a guide wire which extends through the guide wire lumen.

24. The delivery system of claim 23, wherein the delivery catheter is slidable disposed for longitudinal movement along the guide wire.

25. The delivery system of claim 23, wherein the guide wire is fixed to the delivery catheter.

26. The delivery system of claim 25, wherein the guide wire is fixedly mounted to the filtering device.

27. The delivery system of claim 12, wherein the filtering device is located distal to the delivery lumen.

28. A delivery system for administering therapeutic drugs or substances in a body vessel of a patient, comprising:
a delivery catheter having a proximal end and a distal end and a delivery lumen for receiving the cannula of a syringe and a guide wire lumen for receiving a guide wire, the delivery lumen having a distal end through which the cannula extends for the administration of a therapeutic drug or substance to the body vessel;
a filtering device associated with the drug delivery catheter for capturing particles which may be entrained in the body fluid passing through the body vessel;
a guide wire disposed within the guide wire lumen; and
a delivery sheath adapted to extend co-axially over the delivery catheter and the filtering device when the delivery system is being advanced within the body vessels of the patient.

29. The delivery system of claim 28, wherein the filtering device includes an expandable cage and a filter element attached thereto.

30. The delivery system of claim 29, wherein the expandable cage is made from a self-expanding material.

31. The delivery system of claim 28, further including a plurality of delivery lumens.

32. The delivery system of claim 29, wherein the expandable cage includes a proximal strut which extends outwardly in a radial fashion when placed in the expanded position, the end of the delivery lumen being attached thereto.

33. The delivery system of claim 28, wherein the delivery catheter is slidable disposed for longitudinal movement along the guide wire.

34. The delivery system of claim 28, wherein the guide wire is fixed to the delivery catheter.

35. The delivery system of claim 34, wherein the guide wire is fixedly mounted to the filtering device.

36. The delivery system of claim 28, wherein the filtering device is located distal to the filter element.

37. A method for administering therapeutic drugs or substances intraluminally in a body vessel of a patient, comprising:
providing a delivery system including a delivery catheter having a proximal end and a distal end and a delivery lumen for receiving the cannula of a drug delivery device, the delivery lumen having a distal end through which a cannula extends for the administration of a therapeutic drug or substance to the body vessel and an filtering device associated with the delivery catheter for capturing unwanted particles which may be entrained in the body fluid passing through the body vessel;
advancing the delivery catheter intraluminally through the patient to position the end of the delivery lumen near or in contact with the location of the body vessel where the drug or substance is to be administered;
deploying the filtering device within the body vessel;
advancing the cannula of a drug delivery device through the delivery lumen placing the end of the cannula near or into the body vessel; and
administering the drug or substance to the body vessel.

38. The method of claim 37, further including:
collapsing the filtering device for removal from the body vessel.

39. The method of claim 37, wherein the filtering device is located at a distal location from the distal end of the delivery lumen.

40. The method of claim 37, further including a delivery sheath adapted to extend coaxially over the delivery catheter and the filtering device in a delivery position, the delivery sheath being retractable from the filtering device when the filtering device is to be deployed in the body vessel.

41. The method of claim 40, wherein the filtering device includes an expandable cage and a filter element attached to the expandable cage, the expandable cage being biased in an expanded position and collapsible to a delivery position.

42. The method of claim 37, wherein the filtering device is movable between an unexpanded position and an expanded position, the delivery lumen being attached to the filtering device and being positioned near or in contact with the body vessel when the filtering device is deployed in its expanded position.

43. The method of claim 42, wherein the filtering device includes an expandable cage and a filter element attached thereto, the expandable cage including a proximal strut which expands in an outward radial fashion when placed in the expanded position, the delivery lumen being attached to the proximal strut.

44. The method of claim 43, wherein the delivery catheter has plurality of delivery lumens and the expandable case has a plurality of proximal struts, each delivery lumen being attached to an individual proximal strut.

45. The method of claim 37, wherein the delivery catheter includes a guide wire lumen and the delivery system includes a guide wire disposed through the guide wire lumen.

46. The method of claim 45, wherein the delivery catheter is slidably disposed for longitudinal movement along the guide wire.

47. The method of claim 45, wherein the guide wire is fixed to the delivery catheter.

48. The method of claim 47, wherein the guide wire is fixedly mounted to the filtering device.

49. The method of claim 46, wherein the guide wire is advanced initially into the location where the drug or substance is to be administered, thereafter the delivery system being advanced along the guide wire to position the delivery catheter to the location of the body lumen where the drug or substance is to be administered.

50. The method of claim 47, wherein the guide wire and delivery system are simultaneously advanced intraluminally through the patient to position the end of the delivery lumen near or in contact with the location of the body vessel where the drug or substance is to be administered.

51. The method of claim 50, further including:
collapsing and removing the filtering device from the body vessel.

52. The method of claim 37, wherein the filtering device includes an expandable cage and a filter element attached thereto, the expandable cage having a proximal end and a distal end, the expandable cage being expandable to a deployed position by the application of an external force to either or both of the distal and proximal ends of the expandable cage.

53. The delivery system of claim 37, wherein the delivery lumen is adapted to receive the cannula of a drug delivery device which utilizes high pressure jet injection for administering the drug or therapeutic substance to the body vessel.

54. The method of claim 37, wherein the deployment of the filtering device causes a portion of the vessel wall to stretch in order to receive an injection of therapeutic drug or substance.

55. The method of claim 37, wherein the cannula of the drug delivery device pierces the body vessel in order to inject the drug or therapeutic substance into the body vessel.
